(19) United States
(12) Plant Patent Application Publication
(10) Pub. No.: US 2016/0316595 P1
(43) Pub. Date: Oct. 27, 2016

(54) Columnar apple tree named 'A 68-173'

(50) ;

(71) Applicant: Peter Braun, Geisenheim (DE)

(72) Inventor: Peter Braun, Geisenheim (DE)

(21) Appl. No.: 14/545,321

(22) Filed: Apr. 22, 2015

(51) Int. Cl. A01H 5/00

(52) U.S. Cl. PLT/175

(57) ABSTRACT

A new and distinct apple tree variety is provided which exhibits a columnar tree type. The fruit has a very juicy flesh with a good sweet/sour balance, providing a desirable dessert apple.

### BOTANICAL CLASSIFICATION

Malus genus

### VARIETAL DENOMINATION

‘A 68-173’

### BACKGROUND OF THE INVENTION

The present invention comprises a new and distinct cultivar of apple tree of the Malus genus and known by the varietal name ‘A 68-173’. The new variety was bred in Geisenheim, Germany.

The following characteristics distinguish the new variety from other varieties known to the breeder:
Columnar tree type;
Good drought tolerance; and
Tolerance to mildew and scab.

The new variety has been trial and field tested and has been found to retain its distinctive characteristics and remain true to type through successive asexual propagations.

### DESCRIPTION OF THE DRAWINGS

The accompanying photographic drawings illustrate the new cultivar, with the color being as nearly true as is possible with color illustrations of this type. It should be noted that colors may vary with growing conditions and time of year:

FIG. 1 shows a close-up view of the fruits of the new variety;

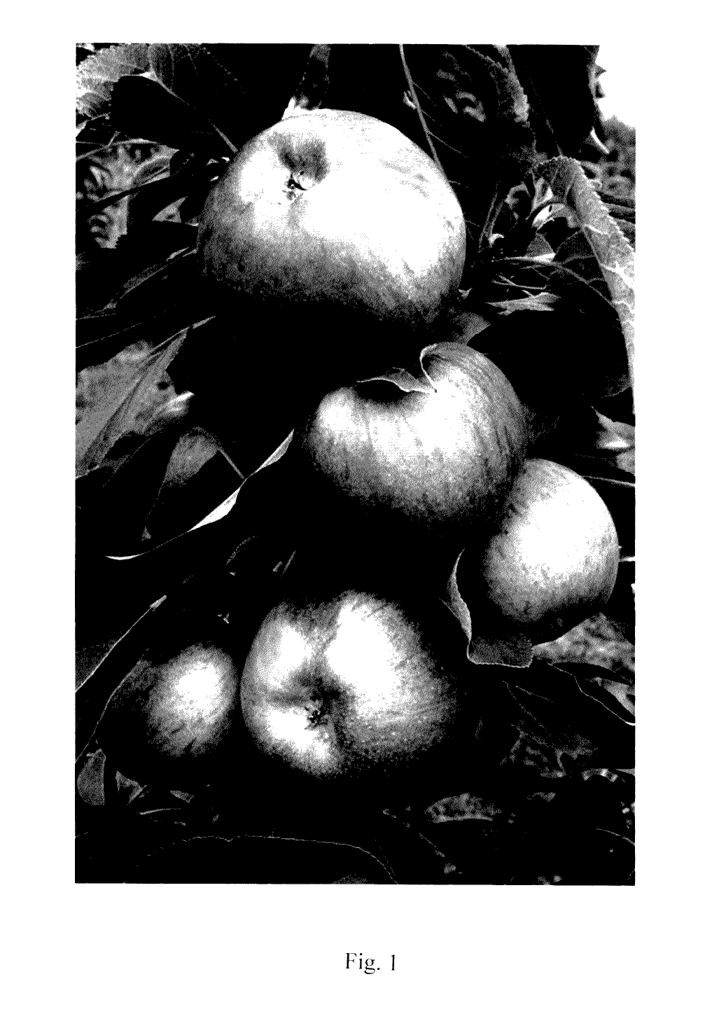

FIG. 2 shows a close-up view of the fruits of the new variety;

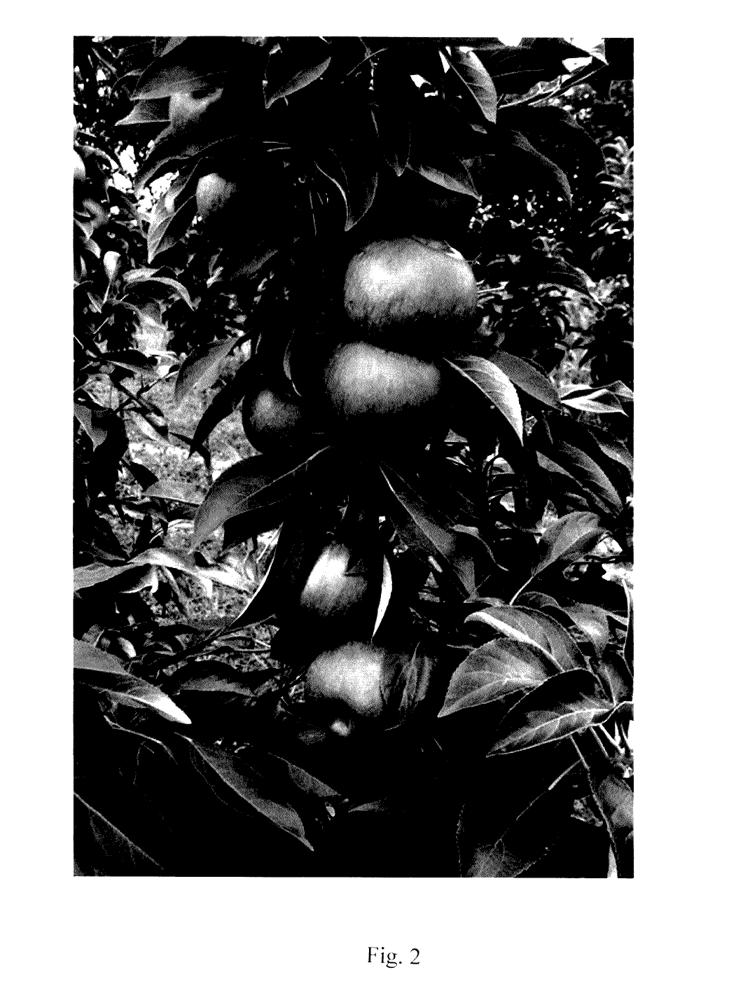

FIG. 3 is a photograph of multiple trees of the new variety;

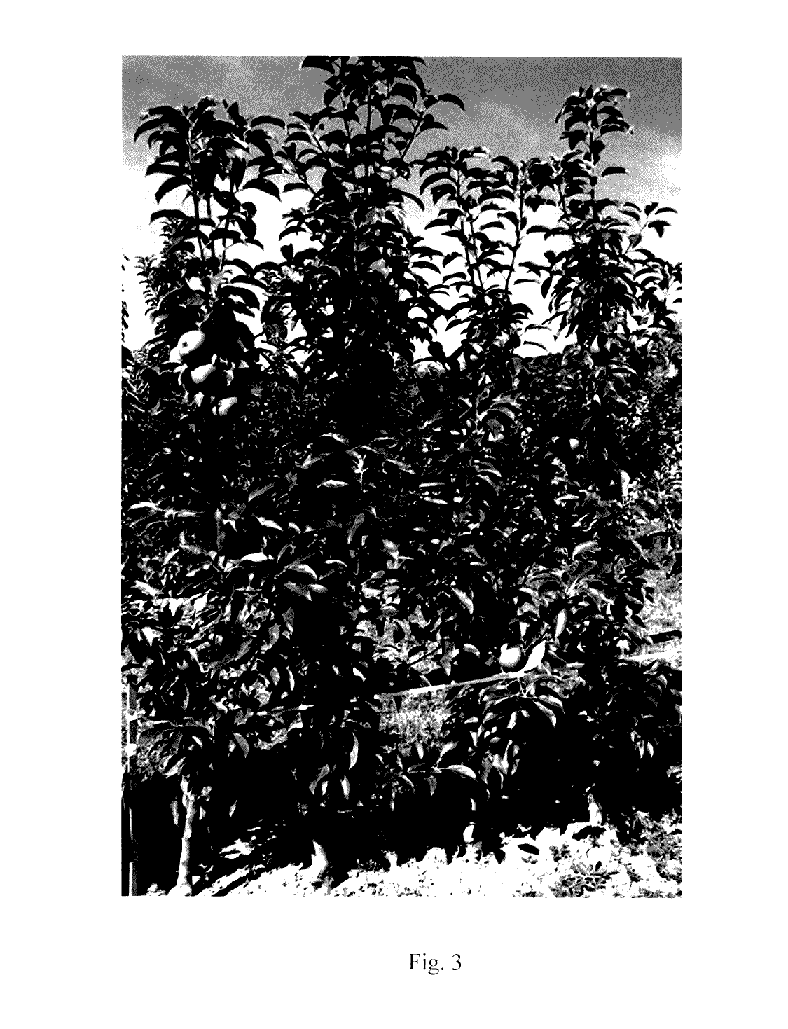

FIG. 4 shows the blossoms and leaves of the new variety; and

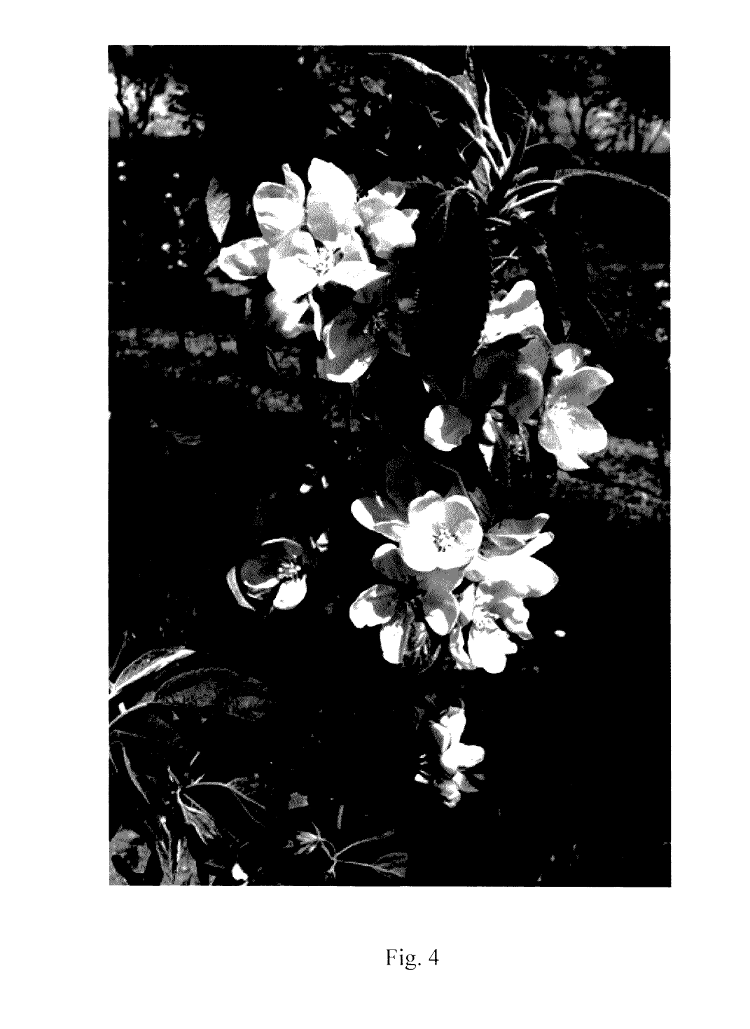

FIG. 5 shows the blossoms of the new variety.

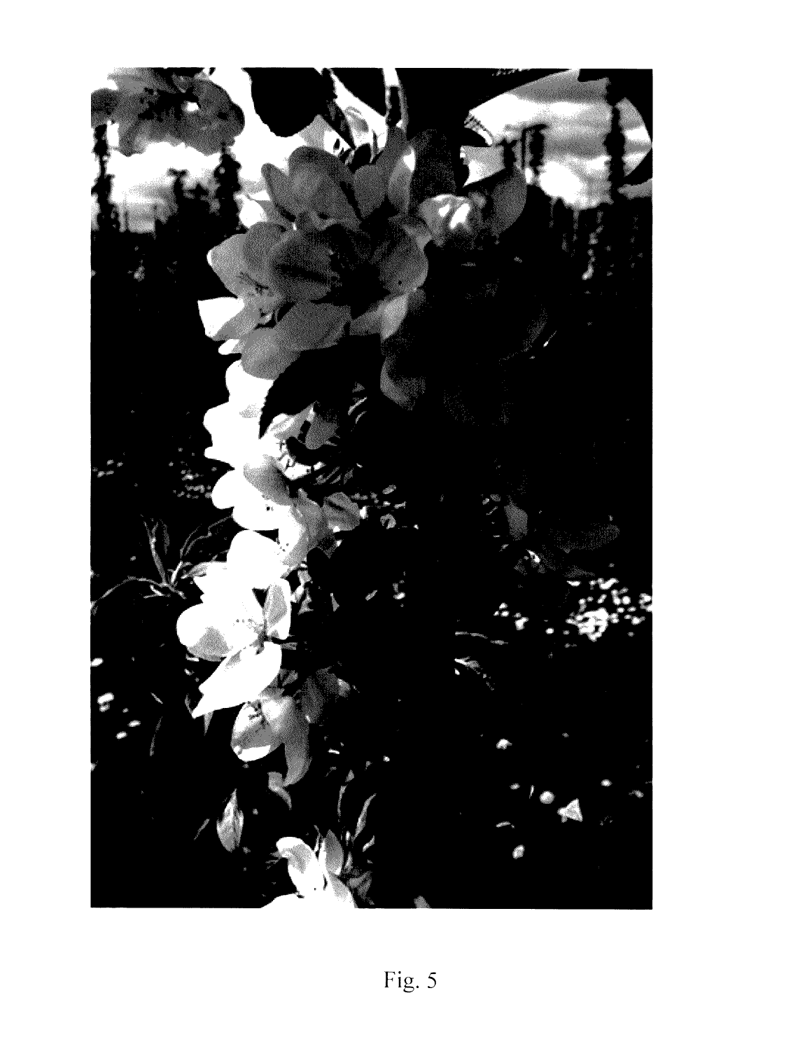

### DESCRIPTION OF THE PLANT

The following detailed description sets forth the characteristics of the new cultivar. The new variety was grown under natural field conditions in Oregon. Color references are primarily to the R.H.S. Colour Chart of The Royal Horticultural Society of London and were identified under natural light.

### TREE

Fruit:
   Maturity when described.—Eating maturity — after 2 months in common storage.
   Date of picking.—Sep. 20, 2013.
   Size.—Axial diameter: Average of 7 cm. Transverse diameter: Average of 9 cm.
   Form.—Slightly flattened.
   Cavity.—Shape: Funnel-shaped, slightly Russet. Depth: 27 mm. Breadth: 18 mm.
   Basin.—Shape: Shallow/wide, with irregularities. Depth: 10 mm. Width: 17 mm.
   Calyx.—Irregular, with some bumps.
Skin:
   Thickness.—Medium.
   Texture.—Firm and juicy.
   Tendency to crack.—Absent.
   Color.—Bicolored; 40% red 42A color with a green background.
   Ground color.—144B.
Flesh:
   Aroma.—Slight.
   Color.—White; 155A.
   Texture.—Fine, with some dents.
   Eating quality.—Very juicy with a combined tart/sweet flavor.
Core:
   Bundle area.—35 mm in size, with tight seed carpels.
   Bundle.—Lightly defined vascular strands.
   Calyx tube.—Narrow and funnel-shaped.
   Depth of tube to shoulder.—14 mm; calyx tube itself is about 7 mm long.
   Styles.—Persistent as dry residues.
   Stamens.—Persistent as dry residues.
   Seed cells.—Wall: Smooth. Depth: 10 mm. Breadth: 5 mm. Longitudinal section: 9 mm.
Seeds:
   Number perfect.—10.
   Number in one cell.—2.
   Length.—10 mm.
   Breadth.—5 mm.
   Form.—Oval.
   Color.—177B.
Stem:
   Length.—15 mm.
   Width.—3 mm on average.
   Color.—191A.
Use: High quality, dessert variety for fresh consumption.
Keeping quality: Moderate, can be stored for 12 weeks and hold firmness Harvesting lasts for 4 weeks.
Drought tolerance: Good
Disease resistance: Tolerant to mildew and apple scab.

I claim:

1. A new and distinct variety of columnar apple tree substantially as is herein described and illustrated.
